(19) United States
(12) Patent Application Publication
(10) Pub. No.: US 2004/0033529 A1
(43) Pub. Date: Feb. 19, 2004

(54) Stabilized composition of troponin for immunoassays and method of preparation of such a stabilized composition

(75) Inventor: Denis Robert Marie Riochet, Bois Colombes (FR)

Correspondence Address: JACOBSON HOLMAN PLLC, 400 SEVENTH STREET N.W., SUITE 600, WASHINGTON, DC, 20004, US

(73) Assignee: Bio-Rad Pasteur, Marnes La Coquette (FR)

(21) Appl. No.: 10/394,657

(22) Filed: Mar. 24, 2003

Related U.S. Application Data
(63) Continuation of application No. 09/024,888, filed on Feb. 17, 1998, now abandoned.
Application No. 09/024,888 is a continuation of application No. 08/648,295, filed on May 15, 1996, now abandoned.

(30) Foreign Application Priority Data
May 16, 1995 (FR) 95 05 788

(51) Int. Cl. G01N 33/53

(52) U.S. Cl. 435/7.1

(57) ABSTRACT

The present invention relates to stabilized compositions of troponin capable of serving as standard and/or control in immunoassays intended for assaying cardiac and/or skeletal troponin(s) in the blood serum or blood plasma of humans or animals. These stabilized compositions comprise, in aqueous solution, troponin I, troponin T and troponin C in the form of an I-T-C ternary complex.

The invention also relates to a method of preparation of the stabilized compositions of troponin.

The present invention relates to a stabilized composition of troponin capable of serving as standard and/or control in immunoassays intended for assaying cardiac and/or skeletal troponin(s) in the blood serum or blood plasma of humans or animals, as well as to a method of preparation of such a composition.

Troponin is known to be a myofibrillar protein complex consisting of three proteins, troponins I, T and C. This protein complex enables a contribution to be made to the regulation of muscle contraction by Ca²⁺ ions, by interacting with myosin and actin. More precisely, it is known that, when a nerve impulse arrives at the motor end plate of a muscle, there is generation of an action potential which is transmitted to the sarcoplasmic reticulum. Ca²⁺ is then liberated into the cytosol and binds to troponin C, which gives rise to a reinforcement of the interaction between troponin I and troponin C and consequently to a change in conformation of the troponin I-T-C complex. There is then liberation of the actin-myosin interaction sites, permitting the contractional movement of the muscle.

When the muscle is damaged, either during a myocardial infarction in the cardiac muscle or during prolonged physical exertion in the case of skeletal muscle, the contractile proteins thus liberated appear more or less rapidly in the bloodstream.

Thus, the assay of troponin for the early diagnosis of myocardial infarction has recently been recommended, both that of troponin T in Circulation 83, 902-912 and that of troponin I in Am. Heart J. 110 1333-44 (19B7) and Molecular Immunology 29 (2), 271-278 (1992). Similarly, the assay of cardiac troponin T for measuring the success of thrombolytic therapy following a myocardial infarction has been proposed in Br Heart J 71, 242-248, (1994), as well as the assay of skeletal troponin I for measuring muscle damage (Abstract No. 35 of the American Association for Clinical Chemistry, 46th National Meeting, New Orleans, Jul. 17-21, 1994). It should be noted that assay of the different cardiac and skeletal troponins is nowadays a very useful means for the diagnosis of human and animal pathologies.

It is well known that the immunoassays performed in biological analytical laboratories require the manufacturer to supply, besides the reagents needed for the assay (that is to say antibodies, labelled or otherwise, visualizing agents and dilution solutions), a standard of the compound to be determined which, employed under conditions similar to those of the sample under study, will serve as reference for calculation of the results and/or as positive control.

To obtain the standard and/or the control of the compound to be determined, it is possible to use the said purified compound in lyophilized form (accompanied by a solvent in which the compound will be dissolved by the user before use) or in ready-to-use form.

Since biological reagents are unstable, the standard or control solutions prepared from lyophilisate are frozen as single doses and stored at −80° C. It was found, moreover, that these solutions were not stable for more than a few hours at +4° C., even if protease inhibitors or antibacterial agents were added to them. Hence this obliges users to prepare their calibration solutions at the time of use.

The patent application published under Number FR-A-2,701,954, which discloses a stabilized composition of troponin I or T for immunoassay, is known in the prior art, the said composition being characterized in that it consists of an aqueous solution containing troponin I or troponin T mixed with troponin C, and in particular in proportions of 1 to 10 molar equivalents of troponin C per equivalent of troponin I or T, and CaCl₂. This technique enables more or less dilute standard solutions of troponin I or T to be stored for several days at +4° C.

The present invention on the one hand enables standard or control solutions to be obtained which are stable for several days at +4° C., and on the other hand has considerable advantages in respect of both the cost of manufacture and that of use. It makes possible, in effect, an easy and convenient use of the solutions for the assay of one or several parameters, simultaneously or otherwise. The Applicant demonstrated, surprisingly, that the ternary complex formed by troponin I, troponin T and troponin C mixed was stable in solution, and that the stabilized solutions of troponin thereby obtained, whose specificity and sensitivity remained unchanged relative to solutions of purified components, could be used as standard and/or control in diagnostic tests in vitro of one or more troponins, simultaneously if necessary.

The subject of the invention is a stabilized composition of troponin which comprises, in aqueous solution, troponin I, troponin T and troponin C in the form of an I-T-C ternary complex.

The troponins I, T and C may be of human or animal origin, and may be, more specifically, of cardiac and/or muscle origin.

The troponins I, T and C are obtained either from an extract of ground preparation of heart or muscle, or from a mixture of the three troponins I, T and C previously purified.

It is naturally preferable, for reasons of cost of manufacture, to prepare the stabilized compositions of troponins I, T and C according to the invention from crude extract of heart or of muscle.

It is possible, depending on the origin of the mixture of the three troponins, to obtain more or less varied amounts of the said troponins in the composition. Preferably, the solutions according to the invention comprise an equimolar amount of troponin I, troponin T and troponin C, in order to obtain a maximum amount of I-T-C ternary complexes formed.

This I-T-C ternary complex is at the basis of the stability of the troponin composition according to the invention. In accordance with the method of preparation of the troponins, bivalent positive ions, in particular calcium and magnesium, which may be supplied in the form of calcium chloride or magnesium chloride, may, moreover, be added in order to stabilize the proteins still further with one another.

The concentration of troponins I, T and C in the solutions according to the invention corresponds to that generally used in immunoassays, that is to say it may be between 0.01 ng/ml and 1 μg/ml, and preferably between 0.1 ng/ml and 50 ng/ml.

According to an advantageous variant, the stabilized composition according to the invention preferably comprises a CaCl₂ or MgCl₂ concentration equal to 10² to 5×106 times by weight that of the troponin I-T-C complex, and between 100 μm and 100 mM. As a further preference, the composition according to the invention contains 2 mM CaCl₂.

According to another preferred variant, the stabilized composition according to the invention contains a protein loading in the proportion of 0.2 to 2%, and preferably 0.4 to 2%. This protein loading consists, for example, of bovine albumin, foetal calf serum or normal human serum. As a further preference, normal human serum present at a concentration of 10% is used in the composition, which corresponds to a protein loading of the order of 0.8%.

Preferably also, the stabilized composition according to the invention is buffered to a pH of between 5.5 and 6.5, and, as a further preference, to a pH of 6±0.1.

The buffers which may be used are, for example, imidazole buffer solution, potassium phosphate buffer solution or sodium succinate buffer solution. It will be preferable to use 0.1M sodium succinate buffer solution.

The subject of the invention is also a powdered stabilized composition of troponin, preferably in lyophilized form, optionally comprising a protein loading of 0.2 to 2% and calcium chloride or magnesium chloride.

The compositions according to the invention are useful for calibrating and/or controlling diagnostic tests in vitro for troponin I, troponin T or troponin C, or two or three of the troponins I, T and C.

The subject of the invention is also a method of preparation of a stabilized composition of troponin, which consists in:

either performing an extraction of troponin I, troponin T and troponin C from a ground preparation of human or animal heart or muscle, the extraction taking place in the presence of protease inhibitors and bivalent positive ions, in particular calcium and magnesium, which may be supplied in the form of calcium chloride or magnesium chloride,

dialysing, where appropriate, the extract obtained above against a buffer at a pH advantageously of between 5.5 and 6.5 and comprising protease inhibitors and bivalent positive ions, in particular calcium and magnesium, which may be supplied in the form of calcium chloride or magnesium chloride,

diluting the solution of troponins I, T and C obtained above in a buffer advantageously at a pH of between 5.5 and 6.5 and comprising protease inhibitors, a protein loading and bivalent positive ions, in particular calcium and magnesium, which may be supplied in the form of calcium chloride or magnesium chloride, so as to obtain a suitable concentration of troponin I, T and/or C,

or mixing purified troponins I, T and C in equimolar amounts in a buffer advantageously at a pH of between 5.5 and 6.5 and comprising a protein loading and bivalent positive ions, in particular calcium and magnesium, which may be supplied in the form of calcium chloride or magnesium chloride.

The extraction from a ground preparation of heart or muscle according to the method described in the invention enables stable compositions to be obtained which may be used to assay an analyte such as cardiac troponin I or cardiac troponin T, or used to assay two analytes such as, for example, cardiac troponin I and cardiac troponin T on the same composition.

In what follows, examples of embodiment of the invention and the results of comparative stability tests are described.

The method of preparation of stabilized compositions of troponin according to the invention is illustrated in the examples which follow. In these examples, the concentrations are expressed in terms of the final concentration of the solution to be obtained.

Prior to the protocol used to prepare the stabilized compositions, the following buffers are prepared:

Extraction Buffer:

9M Urea

75 mM Tris-HCl, pH 8

1 mM CaCl₂-

60 mM β-mercaptoethanol

protease inhibitors

Dialysis Buffer:

0.1M sodium succinate, pH 6

2 mM CaCl₂

protease inhibitors

Dilution Buffer:

0.1M sodium succinate, pH 6

normal human serum (NHS), 10%

2 mM CaCl₂

protease inhibitors

The protease inhibitors used in the three buffers mentioned above can be, for example, those chosen from SBTI (Sigma T9003), TLCK (Sigma T7254), pepstatin A (Sigma P4265), PMSF and anticathepsin.

The method employed to perform the extractions from heart or muscle is described in greater detail in American Heart Journal, Clinical Investigations, June 1987, volume 113, No.6-“Cardiac-specific troponin-I radioimmunoassay in the diagnosis of acute myocardial infarction”, page 1334.

### EXAMPLE 1

### Preparation of a Human Cardiac Troponin I-T-C Composition

The mixture of troponins I, T, C is extracted from one gram of ground preparation of human heart in 30 ml of extraction buffer described above. The mixture is stirred for 15 minutes using a bar magnet before being centrifuged for 20 minutes at 10,000 g (at +10° C.). The supernatant is recovered and dialysed against the dialysis buffer for 2 hours, and then overnight at +4° C., changing the buffer. A dilution to 1/50 of the solution obtained in dilution buffer is then performed.

A composition comprising a concentration of 63 ng/ml of troponin I is obtained.

### EXAMPLE 2

### Preparation of a Human Cardiac Troponin I-T-C Composition

The same protocol as that described in Example 1 is employed, but the supernatant is not dialysed after extraction.

A composition comprising a concentration of 82 ng/ml of troponin I is obtained.

### EXAMPLE 3

### Preparation of a Human Cardiac Troponin I-T-C Composition

The same protocol as that of Example 1 is employed, but 4 mM MgCl₂ is added, in addition, to the extraction, dialysis and dilution buffers.

A composition comprising a concentration of 31 ng/ml of troponin I is obtained.

### EXAMPLE 4

### Preparation of a Human Cardiac Troponin I-T-C Composition

The same protocol as that described in Example 2 is employed, but 4 mM MgCl₂ is added, in addition, to the extraction and dilution buffers. A composition comprising a concentration of 46 ng/ml of troponin I is obtained.

### EXAMPLE 5

### Preparation of a Troponin I-T-C Composition from Purified Troponins

10 μl of troponin I solution at a concentration of 10 μg/ml, 10 μl of troponin C solution at a concentration of 10 μg/ml and 20 μl of troponin T solution at a concentration of 5 μg/ml are introduced into 960 μl of buffer containing sodium succinate (0.1 M, pH 6) containing 10% of normal human plasma and 222 μg of CaCl₂.2H₂O. It is preferable to perform these operations in a sterile environment using troponin I, troponin T and troponin C solutions sterilized, for example, by passing them through a filter of pore diameter 0.22 μm.

The solution obtained, having a concentration of troponin I in the region of 100 ng/ml, of troponin T of 100 ng/ml and of troponin C of 100 ng/ml, is then used to prepare a series of dilutions of 0.1 to 50 ng/ml of troponin I in storage buffer (dilution buffer with the addition of antibacterial agents to a concentration of 1% final). Identical series may be prepared for troponins T and C.

In order to perform comparative stability tests with compositions of purified troponins, the compositions prepared in Examples 1 to 4 are distributed in the following manner:

under sterile conditions, into sterile Nalgene® vials for the series which will be stored in the form of solutions,

under sterile conditions, into sterile glass vials for the series which will be lyophilized.

Stabilized compositions of troponin according to the invention may also be prepared from muscle of human origin, or from heart or muscle of animal origin, using the protocol described in the reference cited above (American Heart Journal, Clinical Investigations, June 1987, Volume 113, No. 6, “Cardiac-specific troponin-I radioimmunoassay in the diagnosis of acute myocardial infarction”. page 1334).

Implementation of Comparative Stability Tests:

From concentrated solutions described in Examples 1 to 4, troponin solutions comprising a troponin I concentration of the order of 1 ng/ml are prepared. To this end, the solutions are diluted appropriately in a storage buffer (dilution buffer with the addition of Kathon® to a concentration of 1% final). Kathon®, an antibacterial agent marketed by the company Haas, consists of 5-chloro-2-methyl-4-isothiazolin-3-one and 2-methyl-4-isothiazolin-3-one (1.5%).

For each example, vials of liquid and of lyophilisate are made available.

The vials of lyophilisate are rehydrated so as to monitor their stability after reconstitution. Day 0 (D0) corresponds to the day of rehydration. In Table I, these vials are designated Lyoph.liq.

The vials of liquid are stored at 4° C. and monitored for stability. Day 0 (D0) corresponds to the day of preparation. In Table II, these vials are designated Liq.

A quality control is performed with a lyophilized reference comprising purified troponin I in normal human serum (designated “Reference” in Table I). This reference is prepared and used at the required time.

In each test, the points of the series which are prepared are read on a lyophilized troponin I series such as the one prepared in the patent application published under No. FR-A-2,701,954 (5 molar equivalents of troponin C per equivalent of troponin I and CaCl₂). By way of comparison, a solution of liquid troponin I prepared according to the invention described in the patent application published under No. FR-A-2,701,954 is monitored for stability at 4° C. This comparative solution is designated “FR-A-2,701,954” in Tables I and II.

Tables I and II give, respectively, the results for stability of the compositions according to the invention, in lyophilized and liquid form, in comparison with the reference compositions mentioned above (the concentration measurements are expressed in ng/ml).

The assay of the troponins according to the method is described in Patent Application FR-A-2,701,954.

It should be noted that the initial concentrations of the solutions measured are not identical. As a result, to arrive at a conclusion regarding stability, the fluctuations of the concentrations observed over time for the same solution were considered.

TABLE I

| | D0 | D + 10 | D + 30 |
|---|---|---|---|
| | Lyoph.liq. | Lyoph.liq. | Lyoph.liq |
| Reference | 0.84 | 0.88 | 0.90 |
| FR-2, 701, 954 | 0.97 | 0.80 | 0.69 |
| Example 1 | 1.2 | 1.23 | 1.26 |
| Example 2 | 1.15 | 1.03 | 1.07 |
| Example 3 | 1.73 | 1.75 | 1.84 |
| Example 4 | 1.48 | 1.48 | 1.55 |

TABLE II

| | D0 | D + 10 | D + 30 |
|---|---|---|---|
| | Liq. + 4° C. | Liq. + 4° C. | Liq. + 4° C. |
| Reference | 0.84 | 0.88 | 0.90 |
| FR-2, 701, 954 | 0.97 | 0.82 | 0.63 |
| Example 1 | 1.18 | 1.23 | 1.1 |
| Example 2 | 1.06 | 1.08 | 0.98 |
| Example 3 | 1.71 | 1.73 | 1.78 |
| Example 4 | 1.64 | 1.73 | 1.68 |

The results of the tests performed demonstrate that the compositions of Examples 1 to 4 according to the invention display a higher stability at one month (D+30) relative to the control FR-2,701,954. The compositions according to the invention are hence all the more stable compared to the standard compositions of purified troponin I which display a stability of only a few hours at 4° C. These results demonstrate convincingly that the I-T-C troponin compositions according to the invention display an enhanced stability, and thus make it possible to be used as standard and/or control in immunoassays intended for assaying cardiac and/or skeletal troponin(s) in the blood serum or blood plasma of humans or animals.

1. A stabilized composition of troponin for immunoassays, containing in an aqueous solution, troponin I, troponin T and troponin C in the form of an I-T-C ternary complex.

2. The composition according to claim 1, comprising bivalent positive ions.

3. The composition according to claim 1, comprising CaCl₂ or MgCl₂.

4. The composition according to claim 3, wherein the concentration of the I-T-C ternary complex is between 0.01 ng/ml and 1 μg/ml, and the CaCl₂ concentration is between 100 μM and 100 mM.

5. The composition according to claim 1, in an aqueous solution buffered to a pH of between 5.5 and 6.5.

6. The composition according to claim 1, said composition comprising a protein loading of 0.2 to 2% by weight.

7. The composition according to claim 1, wherein the protein loading is in the form of normal human serum at a concentration of 10% by volume.

8. The composition according to claim 1, wherein the troponin I, troponin T and troponin C are obtained from the extraction of a ground preparation of human or animal heart.

9. The composition according to claim 1, wherein the troponin I, troponin T and troponin C are obtained from the extraction of a ground preparation of human or animal muscle.

10. The composition according to claim 1, wherein the troponin I, troponin T and troponin C are obtained from a mixture of purified troponin I, purified troponin T and purified troponin C.

11. A powdered composition for the preparation of a composition according to claim 1, said powdered composition containing troponin I, troponin T, troponin C and optionally an additional protein loading of 0.2 to 2% and bivalent positive ions.

12. The composition according to claim 11, in a lyophilized form.

13. A method for calibrating and/or controlling diagnostic tests in vitro for troponin I, troponin T or troponin C, or for two or three of the troponins I, T and C, it being possible for the said troponins to be of cardiac or skeletal origin, said method using a composition according to claim 1.

14. A method of preparation of a composition according to claim 1, which comprises the steps of
performing an extraction of troponin I, troponin T and troponin C on a ground preparation of human or animal heart or muscle, the extraction taking place in the presence of protease inhibitors and bivalent positive ions,
dialysing the extract obtained above, where appropriate, against a buffer comprising bivalent positive ions,
diluting the solution of troponins I, T and C obtained above in a buffer comprising protease inhibitors, a protein loading and bivalent positive ions, so as to obtain a suitable concentration of troponin I, T and/or C.

15. A method of preparation of a composition according to claim 14, wherein the dialysis and the dilution are carried out at a pH of between 5.5 and 6.5.
